(19) United States
(12) Patent Application Publication
(10) Pub. No.: US 2013/0108580 A1
(43) Pub. Date: May 2, 2013

(54) FUSION PROTEIN AND ITS USES

(71) Applicant: Eberhard-Karls-Universitaet Tuebingen Universita, Tuebingen (DE)

(72) Inventors: Christoph Leder, Wendisch Evern (DE); Meinrad Gawaz, Tuebingen (DE); Melanie Ziegler, Herrenberg-Kayh (DE); Konstantinos Stellos, Tuebingen (DE)

(73) Assignee: Eberhard-Karls-Universitaet Tuebingen Universitaetsklinikum, Tuebingen (DE)

(21) Appl. No.: 13/631,497

(22) Filed: Sep. 28, 2012

Related U.S. Application Data
(63) Continuation of application No. PCT/EP2011/054465, filed on Mar. 23, 2011.

(30) Foreign Application Priority Data
Mar. 30, 2010 (DE) 10 2010 013 887.8

(51) Int. Cl. A61K 39/395

(52) U.S. Cl. CPC A61K 38/195
USPC 424/85.1; 530/395; 530/387.3; 536/23.4; 514/20.9; 514/13.3; 514/1.9; 514/16.4

(57) ABSTRACT

The present invention relates to a fusion protein comprising a) a first polypeptide selected from among SDF-1 (stromal cell derived factor-1) or peptidase/protease-resistant variants or fragments thereof which have the CXCR4-/CXCR7- binding function of SDF-1; and b) a second polypeptide which is selected from among GPVI (glycoprotein VI), or the extracellular domain of GPVI, or fragments or variants of the extracellular domain of GPVI which contain the collagen binding function of GPVI, wherein the first polypeptide and the second peptide are linked to one another directly or via a linker molecule. The invention furthermore relates to the use of the fusion protein for treating diseases.

### CROSS REFERENCES TO RELATED APPLICATIONS

This application is a continuation of international patent application PCT/EP2011/054465, filed on Mar. 23, 2011 designating the U.S., which international patent application has been published in German language as WO 2011/120859 A1 and claims priority from German patent application DE 10 201 0 013 887.8, filed on Mar. 30, 2010. The entire contents of these priority applications are incorporated herein by reference

### BACKGROUND OF THE INVENTION

The present invention relates to a fusion protein having therapeutic potential, especially for treating or repairing lesions of vessels, organs or tissues, or for improving hematopoiesis; the invention further relates to a nucleic acid molecule encoding said fusion protein, to a pharmaceutical composition comprising the fusion protein, and to the use of the fusion protein or of the pharmaceutical composition for treating lesions of vessels or tissues, and acute or chronic vascular diseases, or for repairing or remediating same, i.e., for angiogenesis, and for improving/supporting hematopoiesis.

Various findings or diseases—besides physical injuries—can underlie the pathological change in vessels and tissues of the human body. These include, for example, injuries which can occur during implantations of stents or stent grafts, viral or bacterial infections, the occurrence of lesions in diabetes, etc. In arteriosclerosis, too, the degeneration of the arteries occurs as a change in the vascular walls, i.e., more particularly growths and deposits, while in turn various factors can contribute to the development thereof. In the case of, for example, cardiac muscle diseases, changes in the vessels can lead to infarction or a myocarditis.

In general, damage in the vascular wall can lead to loss of integrity in the vascular wall and to subsequent bleeding into surrounding tissue. In order to prevent this, thrombocytes, in conjunction with soluble plasma components, form a hemostatic thrombus, which seals the damage and staunches bleeding. Once a vascular lesion occurs, various cellular and biochemical mechanisms which are necessary for hemostasis are immediately set in motion. In arterial hemostasis, the endothelium also plays a central role via regulation of plasma lipoprotein permeability, via leukocyte adhesion and via secretion of prothrombotic and antithrombotic factors and of vasoactive substances.

The endothelium is the single-layered vascular wall lining which separates the bloodstream from the thrombogenic structures of the subendothelium. In the course of hemostasis, endothelial damage to the vascular wall and the resulting exposed thrombogenic subendothelial matrix lead to the adhesion of quiescent thrombocytes, circulating in the blood, to the now exposed collagen. This initial adhesion process is controlled by thrombocytic membrane glycoprotein receptors, the integrins, and results in a shape change, thrombocyte activation and release of the contents from the storage granules. In this process, the thrombocytic glycoprotein VI (hereinafter also written as “GPVI” for short) interacts directly with the exposed collagen and stabilizes binding. GPVI, as the most important collagen receptor, not only mediates tighter binding directly to collagen, but also mediates the activation of other receptors required for adhesion. Adhesion is followed by the next step in hemostasis, aggregation, which leads to clustering of thrombocytes in the thrombus. Therefore, GPVI, as collagen receptor on the thrombocyte surface, plays a crucial role in the activation of the platelets and is also considered to be a risk factor for myocardial infarction. Owing to the occurrence of such thrombi, the supply of tissue with blood is no longer guaranteed, and so ischemic states of the tissue situated distally to the thrombus can occur.

Cardiovascular diseases, such as angina or myocardial infarction for example, currently still make up about one third of all deaths worldwide. In the case of these diseases, rapid reperfusion of the coronary arteries affected by ischemia is of utmost importance in order to prevent myocardial injury. Once the blood flow in a coronary vessel is reduced, irreversible damage occurs to the myocytes and arrests the functional metabolism in the myocardium, resulting eventually in cell death by necrosis and apoptosis.

The regeneration of tissues, vessels or organs, including myocardium, depends greatly on the recruitment and accumulation of a small population of stem cells at the affected injured or diseased sites. Typically, in response to an injury of the tissues/organs/vessels, stimulation takes place, on the basis of which said stem cells circulate in increased numbers in peripheral blood and adhere in the damaged regions. For instance, it is known that CD34+ stem cells from bone marrow support the integrity of vascular endothelium in that they can differentiate into endothelial cells after adhesion at the affected site.

Specific and directed recruitment of these precursor cells at affected sites would thus represent a preferred tool for supporting natural re-endothelialization. WO 2008/101700 discloses a bispecific fusion protein via which precursor cells can be recruited to tissues/vessels in a specific manner, whereby the fusion protein is bound to injured tissue/vascular sites via a collagen-binding domain (GPVI) and the precursor cells can be recruited via the precursor cell-binding domain.

However, there is still a great need for other alternative products and substances which can achieve improved recruitment of precursor cells or CD34+ cells to affected sites.

### SUMMARY OF THE INVENTION

It is therefore an object of the present invention to provide novel means for treating tissual and vascular diseases, more particularly of coronary vessels, and for repairing injured tissues or vessels.

According to one aspect of the invention, there is provided a fusion protein which comprises a) a first polypeptide selected from SDF-1 (stromal cell-derived factor-1) or variants or fragments thereof which have the CXCR4/CXCR7-binding function of SDF-1; and b) a second polypeptide selected from GPVI (glycoprotein VI), or the extracellular domain of GPVI, or fragments or variants of the extracellular domain of GPVI which have the collagen-binding function of GPVI.

According to another object, there is provided a pharmaceutical composition comprising the fusion protein according to the invention in a pharmaceutically effective amount, optionally in combination with a pharmaceutically acceptable carrier, and by a method for treating diseases in a human, wherein the fusion protein according to the invention or of the pharmaceutical composition comprising said fusion protein is administered in a therapeutically effective amount, especially for treating cardiovascular diseases, for endothelial regeneration in tissues, organs and vessels, and for supporting hematopoiesis and angiogenesis.

The fusion protein according to the invention can bind to collagen via the second polypeptide, viz., the collagen-binding GPVI—or via the extracellular domain of GPVI, or fragments or variants of the extracellular domain of GPVI which have the collagen-binding function of GPVI—and bind CD34+ cells having the receptors of SDF-1, CXCR4 or CXCR7, via the first polypeptide, viz., SDF-1 (stromal cell-derived factor-1) or variants or fragments thereof which have the CXCR4/CXCR-7-binding function of SDF-1, and thus recruit them at sites at which collagen is exposed, i.e., more particularly injured vessels, organs or tissues. The precursor cells recruited at lesions via the fusion protein according to the invention can subsequently differentiate into endothelial cells and be used for regeneration—and ultimately for the treatment of a tissual, organ or vascular disease.

In the present case, a “fusion protein” is understood to mean a hybrid protein or an artificial protein which is obtainable in vitro, but also in vivo, by means of molecular biological or chemical methods known in the prior art by connecting or linking two (or more) (poly)peptides which are otherwise or naturally not connected or linked to one another and also do not otherwise occur naturally. The fusion protein can be prepared by, for example, conjugation of two (or more) polypeptides by means of one or more chemical reagents or by recombinant DNA technologies, i.e., by genetic “linking” of the nucleic acids encoding the proteins. In the latter case, there is the possibility of generating the fusion protein by using customary expression vectors which encode the fusion protein according to the invention. Said expression vectors are introduced into a suitable cell, which then produces the fusion protein.

A “fragment” or “variant” which has or incorporates the binding function of a particular polypeptide is understood herein to mean an amino acid sequence which differs from the wild-type sequence or the sequence specified herein by one or more amino acid substitutions. Such modified amino acid sequences can have “conservative” amino acid substitutions in which the substituted amino acid has the same properties as or similar properties to the replaced amino acid. Similar small changes can also include amino acid deletions and/or insertions. Guidance with regard to determining which and how many amino acid residues can be substituted, inserted or deleted without removing biological activity can, for example, be found by using computer programs known in the prior art. The protein variants or polypeptide variants or fragments thereof that are encompassed herein encompass GPVI proteins or SDF-1 proteins having in each case either the GPVI-binding or the SDF-1-binding property, specifically the identical or a substantially equivalent one. Testing whether a modified GPVI or SDF-1 polypeptide has the binding properties of the unmodified GPVI or SDF-1 polypeptides can be done, for example, in in vitro assays, as will be described below in the present application. Therefore, variants also encompass polypeptides having in each case about 70%, 75%, 80%, 85%, 90%, 92%, 95%, 96%, 97%, 98% or 99% sequence identity with respect to the respective wild-type polypeptide/protein. Determining the percentage sequence identity of two amino acid or nucleic acid sequences can be done according to methods known in the prior art, for example by means of an alignment and calculation via a mathematical algorithm.

“Linker” or “spacer” or “linker molecule”, which are used interchangeably in the present case, is understood in the present invention to mean an amino acid sequence or a nucleic acid sequence encoding it having up to 100 amino acids/bases which is used to link two functional polypeptides, and which, if required, generates a gap between the two functional polypeptides, without exhibiting bioactivity or binding properties toward other molecules. It is therefore a “neutral” sequence which, apart from the functions just described, does not or cannot fulfill other functions.

In a further refinement, a peptidase/protease-resistant SDF-1 variant, more particularly a dipeptidyl peptidase IV-resistant SDF-1 variant, is used as first polypeptide in the fusion protein according to the invention, more particularly a matrix metalloproteinase-resistant variant.

SDF-1 can be cleaved by, inter alia, matrix metalloproteinase (MMP)-2, and it loses its chemotactic bioactivity as a result. This can be prevented by using a modified SDF-1 variant which is MMP-2 resistant, but which retains its chemotactic bioactivity. An example of said SDF-1 variant is described by, for example, Segers et al., (“Local Delivery of Protease-Resistant Stromal Cell Derived Factor-1 for Stem Cell Recruitment after Myocardial Infarction”, Circulation, 2007, 116: 1683-1692); this is termed S-SDF-1 therein, and this is hereby expressly incorporated herein by reference.

The inventors of the present application were able to show in their own experiments that the fusion protein according to the invention was able to bind to soluble collagen and to the CXCR4 receptor of particular cells. It was further shown that the fusion protein was able to stimulate chemotaxis of hematopoietic stem cells. This show that the fusion protein makes it possible to recruit CXCR4+ precursor cells and that the SDF-1 portion of the fusion protein is still functional. Accordingly, the fusion proteins according to the invention provide a simple and specific therapy for the re-endothelialization and restoration of injured vessels, or of any tissue which exposes or uncovers collagen on its surface owing to an injury or other influences, by means of colonization with stem cells and maturation thereof.

The fusion protein according to the invention further provides improved hematopoiesis as required especially in bone marrow ablations or transplantations. The fusion proteins according to the invention can bind to collagen-binding sites for GPVI, which are accessible in the case of the aforementioned interventions after chemotherapy or radiation therapy, accumulate in the bone marrow, and promote and support the repopulation of the bone marrow and blood cell formation at these sites by recruiting precursor cells of stem cells.

Endothelial precursor cells are a circulating, bone marrow-derived cell population of large non-leukocytic cells which are involved in vascular repair and in hemostasis.

The receptor GPVI is the most important receptor in thrombocytes for collagen. GPVI enables platelets to aggregate, secrete, change shape, and activate. Human GPVI contains a signal sequence of 20 amino acids, an extracellular domain of 247 amino acids, and a 21-amino-acid-long transmembrane domain and a 51-amino-acid-long cytoplasmic tail.

Since binding to collagen is mediated via the extracellular domain, it is preferred in one embodiment of the fusion protein according to the invention when the first polypeptide comprises the extracellular portion of GPVI, or fragments or variants of the extracellular domain of GPVI which has the collagen-binding function of GPVI, connected to a dimerizing peptide.

Here, it is advantageous that recourse can be made to, for example, already soluble GPVI, which has been described before in the prior art (see Massberg et al., “Soluble glycoprotein VI dimer inhibits platelet adhesion and aggregation to the injured vessel wall in vivo”, FASEB J. 2004; 18: 397-399, and for the preparation of soluble human GPVI, this publication is hereby expressly incorporated herein by reference.

Soluble GPVI only exhibits affinity to collagen when in dimeric form, for example when connected to the immunoglobulin Fc domain. To generate said soluble GPVI, the extracellular portion of human GPVI can be cloned and connected to the human immunoglobulin Fc domain. This GPVI-Fc protein (hereinafter also termed soluble GPVI-Fc) can, for example, be expressed using adenoviruses via a human HeLa cell line. With such a soluble GPVI-Fc, it was possible to detect adhesion to collagen both in vitro and in vivo.

It will be appreciated by a person skilled in the art that, to fulfill the function according to the invention, viz., for GPVI the binding to collagen, the fusion protein need not necessarily have the full/identical amino acid sequence of the soluble GPVI. On the contrary, the function assigned by the invention to the fusion protein is also fulfilled when the second polypeptide comprises a segment or a sequence variant of the soluble GPVI that still exerts, possibly in attenuated form, the collagen-binding function of GPVI. It is well known that the proteinogenic amino acids are divided into four groups, viz., polar, nonpolar, acidic and basic amino acids. Exchanging a polar amino acid for another polar amino acid, for example glycine for serine, generally leads to little change, if any, in the biological activity of the relevant protein, and so such an amino acid exchange leaves the fusion protein according to the invention largely undisturbed in terms of its function. Against this background, the present invention also comprehends a fusion protein of the kind which comprises, as second polypeptide, a soluble GPVI variant in which one or more than one amino acid of one of the aforementioned amino acid classes is exchanged for another amino acid of the same class. Such a sequence variant is preferably about 70%, more preferably about 80% and most preferably about 90 to 95% homologous to the amino acid sequence of soluble GPVI.

“Fc” stands for “fragment crystallizable”; this fragment is produced by papain cleavage of the IgG molecule, besides the two Fab fragments. The Fc domain consists of the paired CH2 and CH3 domains including the hinge region and contains the part of the immunoglobulin responsible for the dimerization function. Advantageously, recourse can be made here to commercially available human—or mouse—Fc DNA, which either can be isolated by PCR from commercially available cDNA libraries, or which is already present cloned in plasmids, which again can be obtained commercially (for example from Invitrogen, San Diego, USA).

It will be appreciated that an Fc domain fragment or varient can also be used without impairing the function assigned by the invention to the second polypeptide, provided that the fragment or variant still incorporates or has the possibly attenuated dimerization function of an antibody; cf. above statements relating to fragments or variants of GPVI, which equally apply to the fragment or the variant of Fc.

Incidentally, every other molecule comprising a dimerization function is also suitable for being incorporated into the present fusion protein, provided that as a result, the dimerization of GPVI is ensured. It will be evident to a person skilled in the art that the relevant sequence of another dimerization molecule can be incorporated into the fusion protein instead of the Fc portion.

The dimerization molecule is designed with respect to its amino acid sequence such that it comprises a protein segment which is involved in mediating dimerization of two separate proteins or protein subunits. This measure is also simple for a person skilled in the art, since the fine structures, including the amino acid sequences of peptidic dimer complexes, for a multiplicity of proteins are described in detail in the prior art. Known dimer-forming proteins which are known in terms of their sequence and structure include G-proteins, histones, interferon γ, interleukin-2 receptor, Hsp90, tyrosine kinases, IgG molecules, etc. The respective dimerization-mediating domains of the aforementioned proteins can be directly adopted for the preparation of the fusion protein according to the invention. However, it may be perfectly desirable to modify said domains by targeted mutagenesis or else by C- and/or N-terminal addition of single amino acids, so that, for example, the immunological activity of the fusion protein is reduced, more efficient preparation of the fusion protein is made possible, the dimerization function is however largely preserved.

In a further refinement, a variant of the Fc domain or a synthetic Fc fragment is provided which is mutated in the complement- and Fc receptor-binding region such that activation of the immune system is largely reduced and possibly even absent. For example, it is possible to use a Fc fragment in which targeted mutagenesis is carried out at position 331 to exchange a proline for a serine and at amino acid positions 234 to 237 to exchange the tetrapeptide Leu-Leu-Gly-Gly for Ala-Ala-Ala-Ala.

In a further refinement, he first polypeptide has an amino acid sequence corresponding to SEQ ID NO. 1, 2 or 3 from the attached sequence listing.

The amino acid sequence designated SEQ ID NO. 1 shows the sequence of human SDF-1, the amino acid sequence designated SEQ ID NO. 2 shows the isoform SDF-1 alpha (without leader sequence), the amino acid sequence designated SEQ ID NO. 3 shows the isoform SDF-1 beta (without leader sequence). SDF-1 is modified posttranslationally, more particularly the leader sequence, depicted in SEQ ID NO. 8, is cleaved off. SDF-1 is an endogenous chemokine from the group of the CXC motif chemokines, and is also referred to as CXCL12. SDF-1 binds to the chemokine receptors CXCR4 and CXCR7, which belong to the family of G-protein-coupled receptors and which are activated by the binding of SDF-1.

In a further refinement, the second polypeptide has an amino acid sequence corresponding to SEQ ID NO. 4 from the accompanying sequence listing.

The amino acid sequence SEQ ID NO. 4 represents the extracellular domain of human GPVI, inclusive of two further amino acids of the transmembrane domain. It will be appreciated that variants or fragments thereof which have the collagen-binding function of GOVI are also suitable for the purposes of the present invention. A person skilled in the art can, for example, refer to the variants already known in the prior art (as can be found in, for example, the UniProt/SwissProt databases (www.uniprot.org)), or else generate such fragments/variants by means of his or her own obvious experiments, for example by means of amino acid exchanges, deletions, insertions.

As mentioned, in a further refinement, the second polypeptide is the extracellular domain of GPVI, or a fragment or a variant of the extracellular domain of GPVI which has the collagen-binding function of GPVI, and when the second polypeptide is linked to a dimerizing polypeptide, more particularly to an Fc domain of an immunglobulin or a fragment or a variant thereof which has the dimerization function of the Fc domain.

In a further refinement, the Fc domain is a human IgG Fc domain.

In further refinements, the dimerizing peptide is linked to the second polypeptide directly or via a second linker molecule/spacer. In this connection, it is preferred in one embodiment when the second linker molecule has the sequence Glycine-Glycine-Arginine. It will be appreciated that some other sequence can also be used, preferably a sequence similar to the aforementioned one with respect to its polarity. In this case, a person skilled in the art has the knowledge and ability to identify suitable sequences and to appropriately incorporate them into the fusion protein in order to link the dimerizing peptide to the second polypeptide.

In a further refindement, the linker molecule by means of which the first polypeptide is linked to the second polypeptide in a preferred embodiment has the sequence SEQ ID NO. 5 from the accompanying sequence listing. It will be appreciated that other linker molecules can also be used to link the two polypeptides, more particularly those which, compared to the specified linker molecule, lead to little if any change in the biological activity of the relevant fusion protein, and so such an amino acid exchange leaves the fusion protein according to the invention largely undisturbed in its function.

In a refinement, the fusion protein has the amino acid sequence corresponding to SEQ ID NO. 6 or 7. The two sequences differ in that the sequence designated SEQ ID NO. 6 has a sequence for the secretion signal, whereas the sequence corresponding to SEQ ID NO. 7 does not comprise said sequence.

The invention further provides a nucleic acid molecule comprising a sequence selected from the group:
a) the nucleic acid sequence encoding the fusion protein corresponding to SEQ ID NO. 6 or 7, or a variant thereof encoding the same polypeptide according to the degeneracy of the genetic code;
b) a nucleic acid sequence encoding a polypeptide which has at least 70% sequence homology with the polypeptide encoded by SEQ ID NO. 6, wherein the nucleic acid sequence encodes a polypeptide comprising, from its N-terminus to its C-terminus, SDF-1, a nucleic acid sequence encoding a first linker molecule, the extracellular domain of GPVI or a variant thereof capable of binding to collagen, a nucleic acid sequence encoding a second linker molecule, and a nucleic acid sequence encoding a dimerizing polypeptide, that is functional to the effect that it enables a protein encoded by the nucleic acid to be expressed in a cell in a form capable of binding to collagen and/or CXCR4 or CXCR7;
c) a polypeptide-encoding nucleic acid having, in the 5′-3′ direction, a first segment encoding SDF-1 or peptidase/protease-resistant variants or fragments thereof incorporating the CXCR4/CXCR7-binding function of SDF-1, a second segment encoding the amino acid sequence Gly-Gly-Gly-Gly-Ser-Gly-Gly-Gly-Gly-Ser-Gly-GlyGly-Gly-Ser, a third segment encoding an extracellular domain of GPVI or a fragment or a variant of the extracellular domain of GPVI which has the collagen-binding function of GPVI, a segment encoding a linker molecule having the sequence Gly-Gly-Arg, and a fourth segment encoding an Fc domain or a functional conservative variant thereof, that is functional to the effect that it enables a protein encoded by the nucleic acid to be expressed in a cell in a form capable of binding to collagen and/or CXCR4 or CXCR7.

The invention moreover provides a vector comprising a nucleic acid according to the invention; the invention further provides a fusion protein encoded by a nucleic acid according to the invention, and a cell expressing the fusion protein according to the invention.

The fusion proteins according to the invention can be prepared using recombinant expression vectors known in the field. In the present case, a “vector”/“expression vector” is understood to mean a replicable DNA construct which is used to express the fusion protein according to the invention from encoding DNA; it comprises a transcription unit comprising an arrangement of one or more genetic elements having a regulatory role in gene expression, for example promoters, operators or enhancers, which are functionally associated with a DNA sequence which encodes the fusion protein according to the invention and which is transcribed into mRNA and translated into the protein, and with appropriate sequences for transcription and for starting translation and stopping translation. The choice of promoter and other regulatory elements varies in general depending on the (host) cell used. In a preferred embodiment, the nucleic acid encoding the fusion protein according to the invention is transfected into a host cell using recombinant DNA techniques; suitable host cells include prokaryotic, yeast or eukaryotic cells, and are readily accessible to a person skilled in the art on the basis of his or her expertise in conjunction with the present description.

To prepare the recombinant fusion protein, the host cells which have been transfected or transformed with an expression vector carrying the nucleic acid encoding the fusion protein according to the invention are cultured under conditions promoting the expression of the fusion protein according to the invention. The fusion protein can then be purified and isolated from the culture medium or the host cells using methods known in the prior art (see in this regard, for example, Sambrook and Russell, Molecular Cloning, A Laboratory Manual, 3rd edition).

In a further refinement, the invention also provides a pharmaceutical composition containing a fusion protein according to the invention in a pharmaceutically effective amount, optionally together with a pharmaceutically acceptable carrier, diluent or excipient, and/or optionally with further pharmaceutically active substances.

Pharmaceutically acceptable carriers having optionally further additives are generally known in the prior art and are described in, for example, Kibbe A., Handbook of Pharmaceutical Excipients, Third Edition, American Pharmaceutical Association and Pharmaceutical Press 2000. According to the invention, additives encompass any compound or composition which is advantageous for therapeutic use of the composition, including salts, binders, solvents, dispersants, and further substances customarily used in connection with the formulation of drugs.

The fusion protein according to the invention can be integrated into an administration procedure suitable for the particular therapy. Examples of administration procedures include parenteral administration, for example intravenous, intradermal, subcutaneous, transdermal, transmucosal administration. Administered to or injected into the patient in a composition prepared according to the invention which comprises the fusion protein according to the invention, the fusion protein accumulates via the GPVI domain in the region of the endothelial lesions, and as a result a SDF-I gradient is produced and stem cells are recruited. This provides an extremely effective tool for treating diseases whose cause is the lesion of vessels, organs or tissues in which thrombogenic subendothelium is exposed as a consequence.

In a further refinement, the pharmaceutical composition according to the invention is prepared for administration via a stent or balloon catheter.

Alternatively, in a further refinement, the composition according to the invention or the fusion protein is coincubated with a stem cell solution—and the stem cells can thus bind to the fusion protein—and the resulting fusion protein—precursor cell conjugates are administered.

More particularly, the present invention also provides a pharmaceutical composition comprising the fusion protein according to the invention in combination with an active ingredient selected from at least one of G-CSF (granulocyte colony stimulating factor) or dipeptidyl peptidase IV inhibitors.

It is known that dipeptidyl peptidases IV inactivate SDF-1, and so a combination preparation composed of fusion protein and dipeptidyl peptidase IV inhibitors prolongs the half life of SDF-1. On the other hand, it is know that G-CSF brings about the mobilization of stem cells. Therefore, the binding rate of precursor cells to the fusion protein can be increased using a combination of the fusion protein and G-CSF.

The invention further provides for a method for treating diseases or for regeneration, wherein the method comprises the step of administering, to a subject in need thereof, a therapeutically effective amount of the fusion protein according to the invention or of the pharmaceutical composition according to the invention. In a refinement of the method, diseased vessels or tissues are treated. In yet another refinement, hematopoiesis and/or angiogenesis is improved.

As elaborated above, by sing the fusion protein according to the invention or a pharmaceutical composition comprising said fusion protein in the method of the invention, it is possible to treat tissues, vessels or organs in which subendothelium is exposed owing to, for example, an injury or disease, and so, firstly, the fusion protein can bind via its GPVI portion to the collagen exposed as a result and, secondly, CXCR4+ cells, i.e., more particularly precursor cells of stem cells, can be recruited at the injured sites via the SDF-1 portion. There, the precursor cells differentiate into endothelial cells and thus contribute to the re-endothelialization or to the healing of the diseased tissue/organ/vessel.

More particularly, it is possible to treat diseases selected from cardiovascular disease, arteriosclerosis, myocarditis, myocardial infarction; furthermore, the fusion protein according to the invention can be used for regeneration of the myocardium, of the blood-brain barrier in chronic progressive multiple sclerosis, for treatment of fibrotic liver sections, of vascular epithelium, especially after stent implantations or in the case of endothelial infections, after bone marrow ablations, or of tissual and vascular wounds in diabetes. Thus it is possible, inter alia, to successfully treat lesions of vessels, for example coronary vessels, vessels supplying the brain, vessels supplying the extremities, connective tissue, bone, and any vessel or tissue comprising collagen.

It will be appreciated that the features mentioned above and the features yet to be further specified below are possible not only in the particular specified combination, but also in other variations or alone, without departing from the scope of the present invention.

### BRIEF DESCRIPTION OF THE DRAWINGS

The invention will now be more particularly elucidated in the example below and in the figures.

FIG. 1 shows the diagrammatic structure of one embodiment of the SDF-I-GPVI fusion protein (A) according to the invention; and the protein expression of said embodiment (B), wherein the three domains of said embodiment of the fusion protein were detected by immunoblot analyses with corresponding antibodies (anti-SDF-I; anti-GPVI; anti-IgG); the amino acid sequence of the embodiment shown diagrammatically in FIG. 1A is shown in (C), inclusive of a secretion signal sequence;

FIG. 2 shows the detection of collagen binding in an ELISA (enzyme-linked immunosorbent assay) (A); wherein said binding was competeable by incubation with soluble collagen (B);

FIG. 3 shows the binding of said embodiment of the fusion protein to CXCR4 on CD14+ monocytes, depicted by means of FACS (flow cytometry) competition analyses; and

FIG. 4 shows the concentration-dependent stimulation of chemotaxis of human hematopoietic stem cells by the fusion protein according to the invention in a Transwell system.

### DESCRIPTION OF PREFERRED EMBODIMENTS

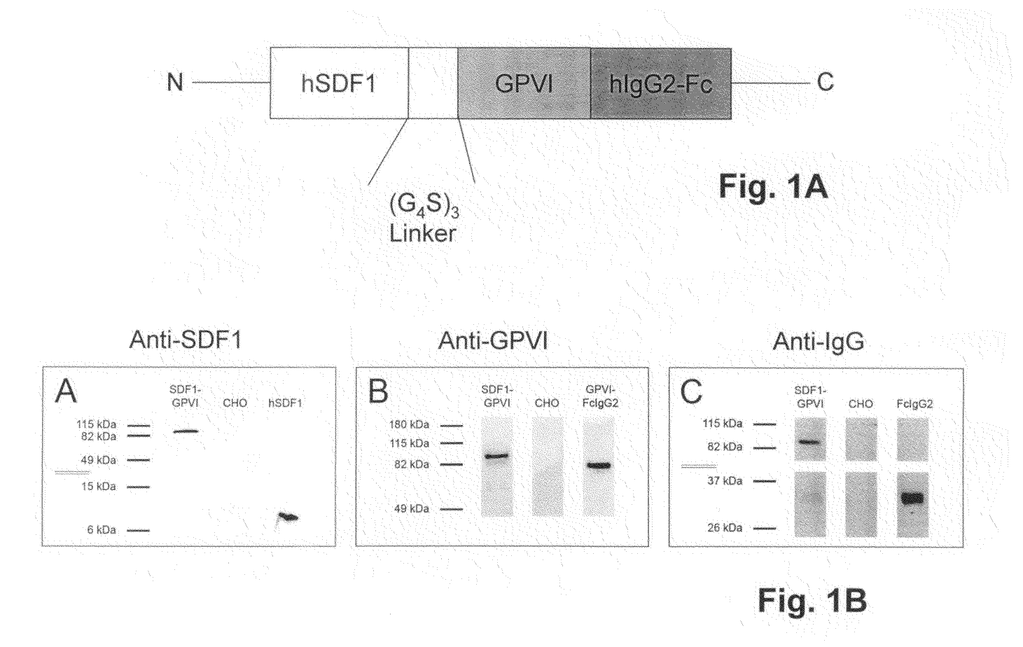

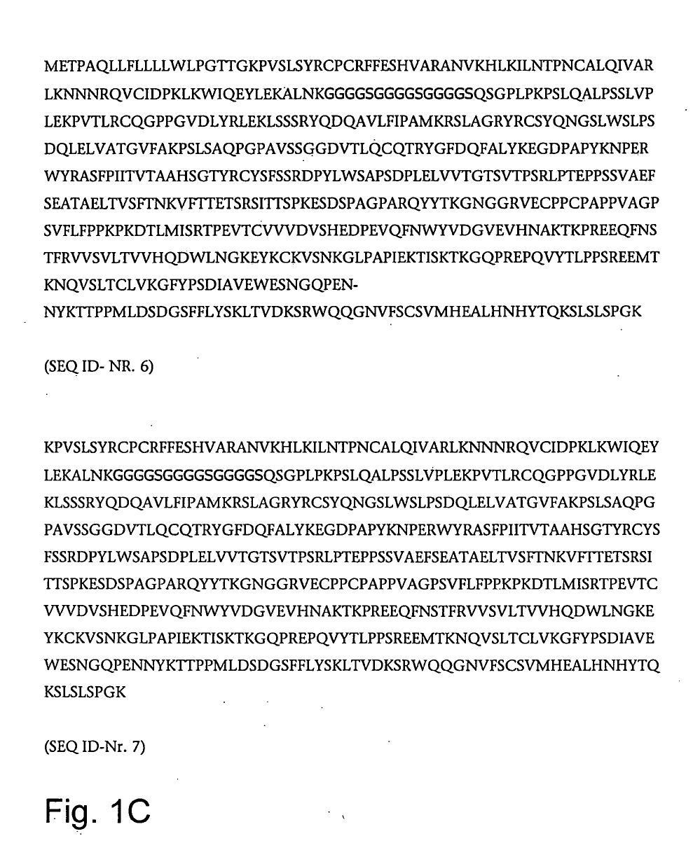

FIG. 1 shows in 1A a diagram of one embodiment of the fusion protein according to the invention, wherein, in succession from the N-terminus to the C-terminus, human SDF-1 is followed by a linker molecule via which SDF-1 is linked to GPVI. In said embodiment, the linker molecule consists of the amino acid chain (Glycine₄Serine)₃. Coupled to the GPVI portion is the human IgG2-Fc portion, which brings about dimerization.

FIG. 1B shows immunoblots, by means of which the expression of the individual components of the fusion protein, as shown diagrammatically in FIG. 1A, was detected. On the left, an anti-SDF-1 antibody (monoclonal anti-human/mouse CXCL12/SDFIalpha antibody; R&D Systems; Minneapolis, USA) was used to detect the SDF-1 portion of the fusion protein; in the middle, an anti-GPVI antibody was used to detect the anti-GPVI portion; and on the right, an anti-IgG antibody was used to detect the IgG2-Fc portion of the fusion protein (first lane in each case). It can be seen that the size of the fusion protein is about 85 kDa. The positive control used was the nonfused polypeptide hSDF-1 for the detection of SDF-1 (third lane), or the GPVI-FcIgG2 construct (for GPVI; third lane) or FcIgG2 alone (for FcIgG2; third lane).

The upper part of FIG. 10 shows the sequence of the fusion protein, which additionally has a 20-amino-acid-long secretion signal sequence (IgK leader sequence) at its 5′ end (SEQ ID NO. 6); SEQ ID NO. 7, shown in the lower part of FIG. 10, shows the fusion protein without said secretion signal sequence. Said secretion signal sequence is responsible for the export of the fusion protein into the cell culture supernatant. Said secretion signal sequence is followed by, from amino acid position 21 to 88, an SDF-1 sequence (without leader) which is 68 amino acids in length. The leader sequence of SDF-1 (MNAKVVVVLV LVLTALCLSDG; SEQ ID NO. 8) is, as mentioned above, not present. This is followed by (position 89 to 103 in SEQ ID NO. 6) a 15-amino-acid-long linker/linker sequence, by means of which the extracellular GPVI domain (position 104 to 352) is linked to the fusion protein. This is in turn followed by a short (3 amino acids) linker (position 353 to 355), by means of which the fusion protein is additionally linked to the IgG2-Fc portion (position 356 to 578).

The embodiment shown in FIG. 1 was generated by means of PCR and primers suitable in each case for the individual segments. The synthesized gene was then cloned into the vector pCDNA5/FRT (Invitrogen). Subsequently, CHO (cells from ovaries of Chinese hamsters) Flp-In cells (Invitrogen) were stably transfected with the construct. The cells expressed the fusion protein into the supernatant, from which it was purified.

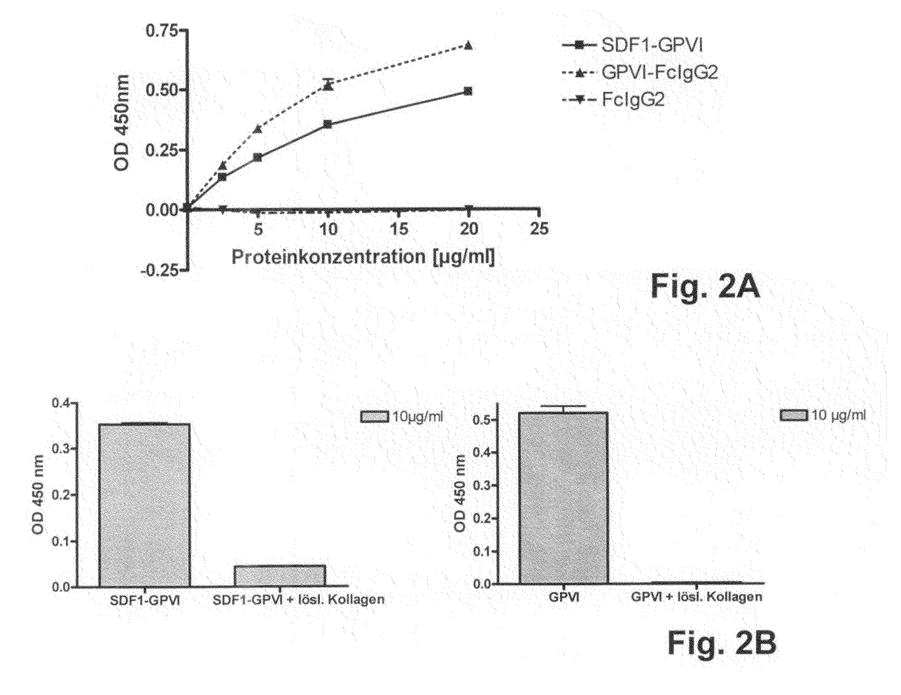

To produce the data shown in FIG. 2, a collagen-GPVI ELISA was carried out. For this purpose, a 96-well plate was coated with 10 μg/ml collagen, blocked with blocking solution, and subsequently incubated with the fusion protein or the appropriate control proteins. Subsequently, detection was carried out using a peroxidase-conjungated anti-human IgG antibody. Thereafter, the values of the binding curves were determined by measuring the wavelength at 450 nm. It can be seen in FIG. 2A that both the fusion protein SDF1-GPVI and the control construct GPVI-FcIgG2 bind to collagen in a concentrationdependent manner, whereas FcIgG2 show no binding.

FIG. 2B showed that binding was almost completely blocked by preincubation of the fusion protein with soluble collagen as competitive inhibitor.

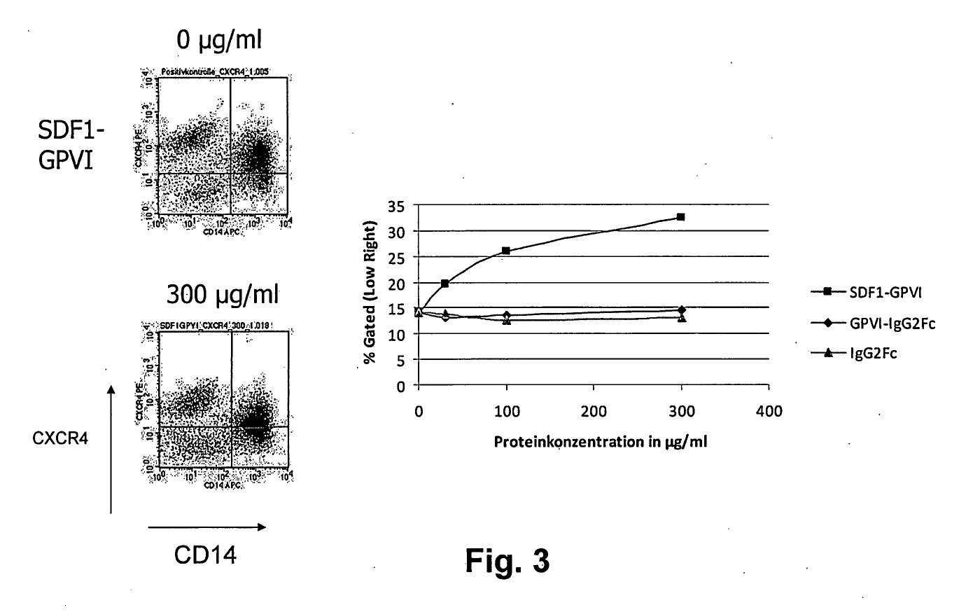

FIG. 3 showed the binding of SDF1-GPVI to CXCR4 by means of a FACS competition assay. For this purpose, monocytes were isolated, since monocytes express CXCR4 on their surface. Said monocytes were incubated with the fusion protein or the appropriate control proteins and subsequently stained with anti-CXCR4-PE labeled antibody (BD Biosciences; catalog number 555974; USA). Owing to the binding of the fusion protein SDF1-GPVI to CXCR4, the antibody is competed out and the anti-CXCR4-PE positively stained cells thus decrease in the subsequent FACS analysis. As a result, the number of analyzed cells in the bottom-right square increases. This showed that the fusion protein SDF1-GPVI binds to CXCR4.

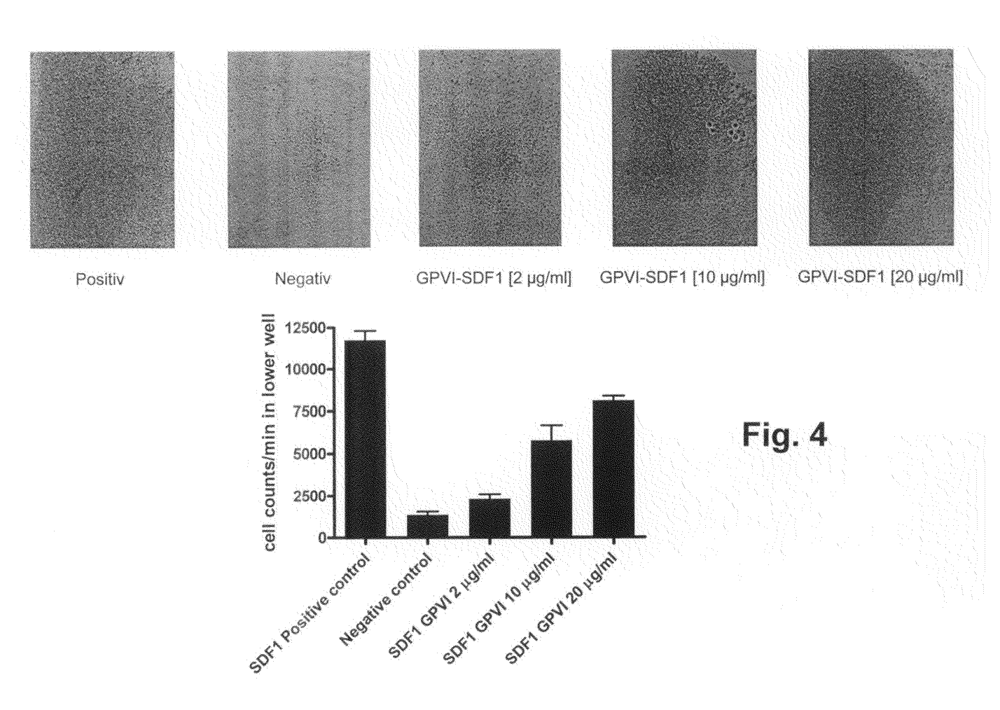

In FIG. 4, the fusion protein was tested with regard to its chemotactic function. For this purpose, the fusion protein SDF1-GPVI, in different concentrations (2 μg/ml; 10 μg/ml; 20 μg/ml), hSDFI as positive control and medium as negative control were introduced into the lower chamber of a Transwell plate. CD34+ hematopoietic stem cells were isolated and 150 000 cells each were added to the upper chamber. After a 6 h incubation at 37° C. in an incubator, the upper chamber was discarded. The cells migrated into the lower chamber were photographed and subsequently the cell count in the lower chamber was determined. For this purpose, the cells from the lower chamber were in each case counted for 1 min by means of FACS. This experiment showed that the fusion protein SDF1-GPVI is chemotactically active.

What is claimed is:

1. A fusion protein comprising
a) a first polypeptide selected from SDF-1 (stromal cell-derived factor-1) or variants or fragments thereof which have the CXCR4/CXCR7-binding function of SDF-1; and
b) a second polypeptide selected from GPVI (glycoprotein VI), or the extracellular domain of GPVI, or fragments or variants of the extracellular domain of GPVI which have the collagen-binding function of GPVI,
wherein the first polypeptide and the second peptide are linked to one another directly or via a first linker molecule.

2. The fusion protein as claimed in claim 1, wherein the first polypeptide has an amino acid sequence selected from SEQ ID NO. 1, 2 or 3, or variants or fragments thereof which have the CXCR4/CXCR7-binding function of SDF-1.

3. The fusion protein as claimed in claim 1, wherein the second polypeptide has an amino acid sequence selected from SEQ ID NO. 4 or 5.

4. The fusion protein as claimed in claim 1, wherein the second polypeptide is the extracellular domain of GPVI, or a fragment or a variant of the extracellular domain of GPVI which has the collagen-binding function of GPVI, and in that the second polypeptide is linked to a dimerizing polypeptide.

5. The fusion protein as claimed in claim 4, wherein the dimerizing polypeptide comprises an Fc domain of an immunoglobulin or a fragment or a variant thereof which has the dimerization function of a human IgG Fc domain.

6. The fusion protein as claimed in claim 4, wherein dimerizing polypeptide is linked to the second polypeptide directly or via a second linker molecule.

7. The fusion protein as claimed in claim 1, wherein the first linker molecule has the sequence SEQ ID NO. 5 from the attached sequence listing.

8. The fusion protein as claimed in claim 1, wherein it has the amino acid sequence corresponding to SEQ ID NO. 6 or 7.

9. A nucleic acid molecule comprising a sequence selected from the group:
a) the nucleic acid sequence encoding the fusion protein corresponding to SEQ ID NO. 6 or 7, or a variant thereof encoding the same fusion protein according to the degeneracy of the genetic code;
b) a nucleic acid sequence encoding a polypeptide which has at least 70% sequence homology with the polypeptide encoded by SEQ ID NO. 6, wherein the nucleic acid sequence encodes a polypeptide comprising, from its N-terminus to its C-terminus, SDF-1, a nucleic acid sequence encoding a first linker molecule, the extracellular domain of GPVI or a variant thereof capable of binding to collagen, a nucleic acid sequence encoding a second linker molecule, and a nucleic acid sequence encoding a dimerizing polypeptide, that is functional to the effect that it enables a protein encoded by the nucleic acid to be expressed in a cell in a form capable of binding to collagen and/or CXCR4 or CXCR7;
c) a polypeptide-encoding nucleic acid having, in the 5′-3′ direction, a first segment encoding SDF-1 or peptidase/protease-resistant variants or fragments thereof incorporating the CXCR4/CXCR7-binding function of SDF-1, a second segment encoding the amino acid sequence Gly-Gly-Gly-Gly-Ser-Gly-Gly-Gly-Gly-Ser-Gly-GlyGly-Gly-Ser, a third segment encoding an extracellular domain of GPVI or a fragment or a variant of the extracellular domain of GPVI which has the collagen-binding function of GPVI, a segment encoding a linker molecule having the sequence Gly-Gly-Arg, and a fourth segment encoding an Fc domain or a functional conservative variant thereof, that is functional to the effect that it enables a protein encoded by the nucleic acid to be expressed in a cell in a form capable of binding to collagen and CXCR4 or CXCR7.

10. A pharmaceutical composition comprising a fusion protein as claimed in claim 1 in a pharmaceutically effective amount, optionally together with a pharmaceutically acceptable carrier, diluent or excipient.

11. The pharmaceutical composition as claimed in claim 10, wherein it is present in combination with an active ingredient selected from at least one of the following: G-CSF (granulocyte colony stimulating factor) or dipeptidyl peptidase IV inhibitors.

12. A method for treating diseases or for regeneration, of vessels or tissues, or for improving hematopoiesis and angiogenesis, wherein a therapeutically active amount of the fusion protein as claimed in claim 1 or a pharmaceutical composition comprising a pharmaceutically effective amount of the fusion protein is administered to a patient in need thereof.

13. The method as claimed in claim 12, wherein the diseases is a cardiovascular disease, arteriosclerosis, myocarditis, myocardial infarction, and dilative cardiomyopathy.

14. The method as claimed in claim 12, wherein the fusion protein as claimed in claim 1 or a pharmaceutical composition comprising a pharmaceutically effective amount of the fusion protein is administered for regeneration of the myocardium, of the blood-brain barrier in chronic progressive multiple sclerosis, of fibrotic liver sections, of vascular epithelium, especially after stent implantations or in the case of endothelial infections, after bone marrow ablations, or of tissual and vascular wounds in diabetes.


SEQUENCE LISTING (8 sequences)

SEQ ID NO: 1 - protein, 93 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNKRFKM

SEQ ID NO: 2 - protein, 68 aa, Homo sapiens
KPVSLSYRCPCRFFESHVARANVKHLKILNTPNCALQIVARLKNNNRQVCIDPKLKWIQEYLEKALNK

SEQ ID NO: 3 - protein, 72 aa, Homo sapiens
KPVSLSYRCPCRFFESHVARANVKHLKILNTPNCALQIVARLKNNNRQVCIDPKLKWIQEYLEKALNKRFKM

SEQ ID NO: 4 - protein, 249 aa, Homo sapiens
QSGPLPKPSLQALPSSLVPLEKPVTLRCQGPPGVDLYRLEKLSSSRYQDQAVLFIPAMKRSLAGRYRCSYQNGSLWSLPSDQLELVATGVFAKPSLSAQPGPAVSSGGDVTLQCQTRYGFDQFALYKEGDPAPYKNPERWYRASFPIITVTAAHSGTYRCYSFSSRDPYLWSAPSDPLELVVTGTSVTPSRLPTEPPSSVAEFSEATAELTVSFTNKVFTTETSRSITTSPKESDSPAGPARQYYTKGN

SEQ ID NO: 5 - protein, 15 aa, Homo sapiens
GGGGSGGGGSGGGGS

SEQ ID NO: 6 - protein, 578 aa, Artificial Sequence [sequence omitted]

SEQ ID NO: 7 - protein, 558 aa, Artificial Sequence
  recombinant fusion protein
KPVSLSYRCPCRFFESHVARANVKHLKILNTPNCALQIVARLKNNNRQVCIDPKLKWIQEYLEKALNKGGGGSGGGGSGGGGSQSGPLPKPSLQALPSSLVPLEKPVTLRCQGPPGVDLYRLEKLSSSRYQDQAVLFIPAMKRSLAGRYRCSYQNGSLWSLPSDQLELVATGVFAKPSLSAQPGPAVSSGGDVTLQCQTRYGFDQFALYKEGDPAPYKNPERWYRASFPIITVTAAHSGTYRCYSFSSRDPYLWSAPSDPLELVVTGTSVTPSRLPTEPPSSVAEFSEATAELTVSFTNKVFTTETSRSITTSPKESDSPAGPARQYYTKGNGGRVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 8 - protein, 21 aa, Homo sapiens
MNAKVVVVLVLVLTALCLSDG